(19) United States
(12) Patent Application Publication
(10) Pub. No.: US 2026/0035318 A1
(43) Pub. Date: Feb. 5, 2026

(54) OXYGEN ASSISTED CRACKING OF HYDROCARBONS IN MOLTEN SALTS

(71) Applicant: Braskem America, Inc., Philadelphia, PA (US)

(72) Inventors: Samuel Lin Tin Chee Leung, Pittsburgh, PA (US); Charles Ambrass, Pittsburgh, PA (US); Goutham Kothamreddy, Pittsburgh, PA (US); Jan Kalfus, Pittsburgh, PA (US); Scott Mitchell, Pittsburgh, PA (US)

(73) Assignee: Braskem America, Inc., Philadelphia, PA (US)

(21) Appl. No.: 19/286,139

(22) Filed: Jul. 30, 2025

Related U.S. Application Data
(60) Provisional application No. 63/677,211, filed on Jul. 30, 2024.

(51) Int. Cl. C07C 4/02

(52) U.S. Cl. CPC C07C 4/025

(57) ABSTRACT

A process for the cracking of a carbon-containing feedstock to produce olefins, aromatics and/or aliphatic includes contacting, in a reactor system, the carbon-containing feedstock with oxygen gas in the presence of a molten salt matrix consisting of a eutectic mixture of alkali metal carbonates, alkali metal hydroxides, alkali metal nitrates, alkali metal halides, alkaline earth metal carbonates, alkaline earth metal hydroxides, alkali earth nitrates, alkaline earth metal halides, rare earth metal carbonates, transition metal carbonates, transition metal hydroxides, transition metal nitrates, transition metal halides, or a mixture of any two or more thereof, to generate an olefin-containing product stream; and collecting an olefin from the olefin-containing product stream; wherein the oxygen is fed with the carbon-containing feedstock in a gas stream comprising from greater than 0 wt % to about 21 wt % oxygen in an inert gas; and the process is conducted in the absence of a catalyst

### CROSS REFERENCE TO RELATED APPLICATIONS

This application claims the benefit of priority to U.S. Provisional Patent Application No. 63/677,211, filed Jul. 30, 2024, which is hereby incorporated by reference, in its entirety for any and all purposes.

### FIELD

The present technology is generally related to the cracking of carbon-containing feedstocks. More specifically, it is related to molten salt compositions and methods of cracking in the presence of oxygen to form olefinic, aromatic and aliphatic monomers from a carbon-containing feedstock.

### BACKGROUND OF THE INVENTION

Pyrolysis is a promising technology that can be used to produce valuable oils and light hydrocarbons in high yields from waste plastic feedstocks. In this regard, ethylene and propylene are the key feedstocks for the production of polyolefins (i.e. polyethylene and polypropylene). The majority of ethylene and propylene is produced by steam cracking, a process in which a fossil-based, hydrocarbon feed (composed primarily of naphtha, liquified petroleum gas (LPG), and/or ethane) is diluted with steam and heated in a furnace in the absence of oxygen to temperatures around 850° C. This process is highly endothermic, typically requiring ˜60 GJ/ton of high value products. Conventional furnaces burn hydrogen (produced from the steam cracking process) and methane to meet this energy requirement, resulting in large emissions of CO₂. The continued dependence on both a fossil-based feedstock and fuel contributes greatly to the emissions of petrochemical operations.

Plastics account for roughly 12% of municipal solid waste in the U.S. and constitute a potential alternative/supplemental hydrocarbon feedstock. Specifically, polyolefins consist of long chains of carbons that, in atomic composition and arrangement, do not differ greatly from conventional fossil-based feedstocks, but do differ in average molecular weight (1000s of g/mol vs. 100-200 g/mol, respectively). Polyolefins, as a feedstock, are attractive because they present a potential opportunity for closed-loop circular plastics.

Waste plastics can contain a number of both organic and inorganic contaminants from both their production (e.g. additives, pigments, metals, etc.) and their subsequent processing (e.g. paper, PVC, water, etc.). Plastics thus cannot be easily used directly as a supplemental or alternative feedstock to a steam cracker for the production of light olefins.

Pyrolysis can be an intermediate processing step to convert polyolefins to pyrolysis oil (or “py-oil”). This process breaks down polyolefins into liquid “py-oil” fractions (i.e., naphtha, diesel), solids (waxes), and lower molecular weight gases. Char/carbon is often a low-value byproduct of the process. The naphtha can then be fed to a conventional cracker to produce light olefins. The process typically occurs around 500° C., as higher temperatures can lead to side reactions, including aromatization. Conventional pyrolysis, however, struggles with fouling (from char) and contaminants (e.g. metals, PVC), which can become entrained in the liquid products, exceeding limits imposed by the steam cracker. As a result, efforts are underway to improve purification of the feed plastic as well as the pyrolysis oil.

### SUMMARY

Oxidative cracking is an attractive route for the direct conversion of carbon-containing feedstocks (e.g., waste plastics, bio-based complex compositions, light alkanes, and municipal solid waste) into value-added basic chemical building blocks (e.g., olefins, oxo-compounds, and aromatics). The oxidative conversion of carbon-containing feedstocks reduces carbon dioxide emissions usually associated with heating requirements in the production of value-added basic chemical building blocks. One way to perform oxycracking relies on the use of a molten salt, and the present inventors have found that by including oxygen with the feedstock, improvements in process efficiency may be achieved.

In one aspect, a process is provided for the cracking of a carbon-containing feedstock to produce olefins, aromatics and aliphatic, the process comprising: contacting, in a reactor system, the carbon-containing feedstock with oxygen gas in the presence of a molten salt matrix consisting of a eutectic mixture of alkali metal carbonates, alkali metal hydroxides, alkali metal nitrates, alkali metal halides, alkaline earth metal carbonates, alkaline earth metal hydroxides, alkali earth nitrates, alkaline earth metal halides, rare earth metal carbonates, transition metal carbonates, transition metal hydroxides, transition metal nitrates, transition metal halides, or a mixture of any two or more thereof, to generate an olefin-containing product stream; and collecting an olefin from the olefin-containing product stream; wherein: the oxygen is fed with the carbon-containing feedstock in a gas stream comprising from greater than 0 wt % to about 21 wt % oxygen in an inert gas; and the process is conducted in the absence of a catalyst.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and may be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein may be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “cracking” refers to a chemical process whereby a feedstock, i.e. complex organic molecules such as long-chain hydrocarbons, carbohydrates, or others are broken down into simpler molecules such as light hydrocarbons, oxygenates, or carbon oxides by the breaking of chemical bonds in the feedstock.

As used herein, the term “thermocracking” refers to a cracking process, whereby the conversion of feedstock to products is achieved by thermal energy transfer, i.e. heating, and, hence, it requires operating at elevated temperatures to proceed.

As used herein, the term “oxycracking” refers to a cracking process that utilizes a combination of thermocracking and oxidation processes, generally applied to the processing of heavy carbon-containing feedstocks, resulting in the formation of lighter hydrocarbons products, plus some amounts of organic oxygenates, CO, CO₂, and H₂O as the co-products.

As used herein, the term “reactor system” refers to where the cracking reaction(s) take place. The process for cracking of hydrocarbons may occur in a single reactor or in at least two reactors in series.

As used herein, the term “carbon-containing feedstock” is not only purely hydrocarbon materials as would typically be associated with the term, including but not limited to petroleum feeds of a wide variety (including fossil fuel oils, naphtha, etc.), but the term also refers to any material having a carbon-containing segment within a plastic (i.e. polymer), biomass, or biowaste that is amenable to cracking by the processes provided herein. The carbon-containing feedstock may contain oxygen in the material, as well as other heteroatoms (N, S, Cl, etc.), colorants, plasticizers, and the like typically associated with polymers.

As used herein, the term a “eutectic” or “eutectic mixture” refers to a homogeneous mixture of substances that melt or solidify at a single temperature that is lower than the melting point of any of the constituents individually. It does not necessarily refer to the lowest melting point that is achievable with any particular mixture of substances, this is the eutectic point for those substances, and it may be part of the eutectic mixture. As long as a mixture of substances melts at a temperature lower than the melting point of any of its constituting pure substances, and forms a single continuous phase, it is a “eutectic” or “eutectic mixture” for the purposes of this disclosure.

It has now been found that the cracking of carbon-containing feedstocks may be conducted using a eutectic mixture of molten salts in the presence of oxygen to form a product stream containing olefinic, aromatic and/or aliphatic compounds.

In this regard, the process according to the present invention presents an alternative means of implementing pyrolysis by incorporating a molten salt media. As used herein, molten salt is added as a kind of “processing agent”, thereby providing multiple benefits including improved heat transfer to the plastic particles as well as contaminant capture. The salt and trapped contaminants can potentially be purified later through physical (e.g. filtration) or chemical (e.g. re-oxidation) means.

The methods may be applied to carbon-containing feedstocks and includes recycling of olefinic, aromatic and/or aliphatic polymers and biopolymers alike. The methods may be applied to pure hydrocarbon feedstock streams, as well as mixed streams, particularly where the hydrocarbon stream is from a mixed waste recycling operation. They hydrocarbons may be of a wide variety including petroleum-based streams of heavy or gaseous compounds, plastics, biomass, or biowaste. The described methods have the potential to deliver improved performance over industry accepted methods such as thermal pyrolysis, thermal-steam cracking, fluid catalytic cracking, and supercritical fluid cracking.

The processes described herein take advantage of autothermal or net exothermic cracking processes where the thermal demands for the process are met by all, or at least part of, the internally generated heat by taking advantage of the exothermic process, such as through combustion of hydrogen and partial combustion of the feed. Other accepted processes in the industry rely entirely on externally generated heat to achieve the desired conversion, and, because of this, are more energy and capital-intensive processes.

The processes described herein also tolerate the presence of acid impurities, such as those containing chloride, bromide, sulfide, and sulfate groups in the feed. The removal of such acid impurities is believed to occur through the absorption of such materials into the eutectic mixture, which is then purified, and the impurities removed. Thus, the process is feedstock flexible and can be used to process mixed plastic waste. The processes may also be operated as a continuous, semi-continuous, or batch processes. Thus, the process offers a high degree of flexibility for its end-user application design and operation. The carbon-containing feedstock may be injected into the reactor at either a bottom (i.e. it flows through the molten salt) or a top (i.e. it comes into contact at a surface of the molten salt) of the reactor.

In a first aspect, a process is provided for the cracking of a carbon-containing feedstock to produce olefins. The process includes contacting, in a reactor system, the carbon-containing feedstock with oxygen gas in the presence of a molten salt matrix consisting of alkali metal carbonates, alkali metal hydroxides, alkali metal nitrates, alkali metal halides, alkaline earth metal carbonates, alkaline earth metal hydroxides, alkali earth nitrates, alkaline earth metal halides, rare earth metal carbonates, transition metal carbonates, transition metal hydroxides, transition metal nitrates, transition metal halides, or a mixture of any two or more thereof, to generate an olefin-containing product stream; and collecting an olefin from the olefin-containing product stream. In the process, the oxygen is fed, with an inert gas diluent, concurrently with the carbon-containing feedstock in the same feed stream or as separate a gas stream. The gas stream contains the oxygen from greater than 0 wt % to about 21 wt % oxygen, and the inert gas may include nitrogen, helium, or argon. The gas stream may be air, in some embodiments. In other embodiments, the gas stream contains the oxygen from greater than 0 wt % to about 8 wt % oxygen in inert gas.

It is also noted, importantly, that the process is to be conducted in the absence of a catalyst. Illustrative catalysts include transition metal, transition metal oxide, rare-earth metal, and rare earth metal oxide catalysts and any combination of two or more thereof. According to an embodiment, the process is also formed in the absence of a glass-forming oxides that include oxides of silicon, boron, and phosphorus.

As noted above, the eutectic mixture melts at a lower temperature than its constituent materials, and it melts at a temperature at which the catalytic reactions may be conducted to form desirable materials from a feedstock. The eutectic mixture may be a mixture of alkali metal carbonates, alkali metal hydroxides, alkali metal nitrates, alkali metal halides, alkaline earth metal carbonates, alkaline earth metal hydroxides, alkali earth nitrates, alkaline earth metal halides, rare earth metal carbonates, transition metal carbonates, transition metal hydroxides, transition metal nitrates, transition metal halides. In some embodiments, the eutectic mixture is one of MnCO₃, FeCO₃, CoCO₃, CaCO₃, BaCO₃, Ce₂(CO₃)₃, KOH, NaOH, Ca(NO₃)₂, KNO₃. Those options, however, are non-limiting examples.

In some embodiments, the eutectic mixture has a melting point of less than about 800° C., or less than about 750° C., or less than about 650° C. This includes melting points from about 250° C. to about 650° C., from about 350° C. to about 550° C., or about 400° C.

In the process, a carbon-containing feedstock is injected into a reactor system where it comes into contact with the eutectic mixture and the co-feed of oxygen. An upper temperature limit within the reactor is defined by the need to preserve a substantial fraction of the carbon-carbon bonds of the feed from the thermo-pyrolytic decomposition. For example, for a polyethylene feed, the limit is defined by a ceiling temperature of about 610° C.

Pyrolysis reactions are known to take place primarily via radical-based reactions; Scheme 1 shows potential reaction pathways and propagation steps that lead to the formation of pyrolysis products. These reactions including a) initiation steps, in which a radical is initially formed by C—C cleavage, b) propagation steps, including b.i) b-scission, b.ii) H-shift, and b.iii) H-transfer steps, and c) termination steps, in which radicals are quenched. In radical-mediated reactions, the propagation steps b) occur at much faster rates than steps a) and c). As such, steps b) determine the product distribution and selectivity towards a given product, while steps a) and c) control the amount of product produced. This concept can be used to determine the role that O₂ plays in increasing yields of lighter products.

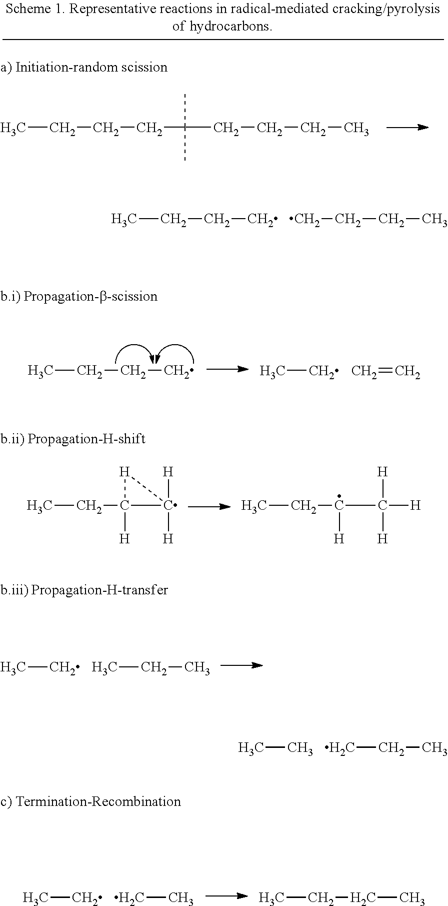

Without being bound by theory, oxypyrolysis likely enhances product yields through a H-abstraction step (Scheme 2), thereby initiating additional radicals that are able to also, like pyrolysis reactions, proceed through a series of propagation steps that result in a similar distribution of products, which in turn increases the yield toward desired light olefins. The results demonstrated here are applicable to a wide range of hydrocarbon feeds, whose only requirement are that they contain C—C bonds that can be cleaved to form a desired product.

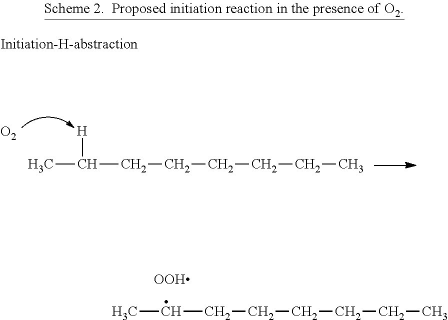

For those familiar with the art, a commercial process designed for thermocracking of polyethylene can operate at a temperature exceeding the ceiling temperature by 50-150° C. in order to achieve economically practical feed-to-monomers conversion rates. In the case of other feeds, the upper temperature limit is a function of the ceiling temperatures of the corresponding monomers, as given in the values in Table 1, reproduced from Stevens, M. P. Polymer Chemistry an Introduction (3rd ed.). New York: Oxford University Press. pp. 193-194 (1999), plus the additional 50-150° C. to achieve practical rates:

TABLE 1
Ceiling temperatures of common hydrocarbon monomers.

| | Ceiling | |
|---|---|---|
| Monomer | Temperature (° C.) | Structure |
| ethylene | 610 | CH₂═CH₂ |
| 1,3-butadiene | 585 | CH₂═CHCH═CH₂ |
| isoprene | 466 | CH₂═C(Me)CH═CH₂ |
| styrene | 395 | PhCH═CH₂ |
| methyl methacrylate | 198 | CH₂═C(Me)CO₂Me |
| isobutylene | 175 | CH₂═CMe₂ |
| α-methylstyrene | 66 | PhC(Me)═CH₂ |

A lower temperature limit is defined by the need to maintain the molten salt in the liquid state.

As noted above, the process provides for the production of olefinic and/or aromatic compounds from a carbon-containing feedstock. Because the carbon-containing feedstock may be from a wide variety of materials for process, including the processing of recycled plastics, biomass, biowaste, mixed plastics, biomass, refinery range hydrocarbons, and/or biowaste, the olefinic and/or aromatic compounds that are produced may include a wide variety of unsaturated compounds such as, but not limited to light olefins, α-olefins, terminal dienes, substituted and unsubstituted aromatic compounds, including single aromatic ring or several aromatic ring compounds.

Illustrative olefinic, aromatic and/or aliphatic compounds are from a wide range of materials. In some embodiments, the olefinic compounds may be from C₂ to C₂₀ olefins, from C₂ to C₁₆ olefins, or from C₂ to C₁₂ olefins, or from subranges of any of these. In some embodiments, the aromatic compounds may be from C₆ to C₁₈ aromatics, or from C₆ to C₁₂ aromatics, or from subranges of any of these. Illustrative olefinic, aromatic and/or aliphatic compounds include, but are not limited to, ethylene, propylene, 1-butylene, 2-methyl-but-1-ene, 1-n-pentene, 2-methyl-pent-1-ene, 3-methyl-pent-1-ene, 1,3-butadiene, 1,3-pentadiene, 1,4-pentadiene, 1,3-hexadiene, 1,4-hexadiene, 1,5-hexadiene, benzene, toluene, ethylbenzene, xylenes, styrene, α-methylstyrene, naphthalene, and anthracene.

As noted herein, the carbon-containing feedstock may include, but is not limited to, any one or more of a refinery range hydrocarbons, a polymer, a biopolymer, biomass, or biowaste. Where the feedstock includes a polymer, illustrative polymers include, but are not limited to those such as polyethylene, polypropylene, polyisobutylene, polybutadiene, polystyrene, poly-α-methylstyrene, polacrylates, poly(meth)acrylates, polyvinyl acetate, and polyvinylchloride. Where the feedstock includes a biopolymer or other bio-based material it may include materials such as fatty acids, triglyceride esters of fatty acids, cellulose, lignin, sugars, animal fat, tissue, and ordure.

Refinery range hydrocarbons are typically defined by their boiling point range fractions. For example, light naphtha has an approximate boiling point range of 25 to 85° C., heavy naphtha has an approximate boiling point range of 85 to 200° C., kerosene has an approximate boiling point range of 170 to 265° C., gas oil has an approximate boiling point range of 175 to 345° C., and heavy residue has an approximate boiling point range of 345 to 656° C. All of these may serve as the feedstock for a refinery range hydrocarbon. In some embodiments where the feedstock includes a refinery range hydrocarbon, it may include asphalt, vacuum resid, heavy residual oil, paraffin wax, lubricating oil, diesel, kerosene, naphtha, or gasoline. In some embodiments, the feedstock may include n-hexane, n-hexadecane, or white mineral oil.

In the process, the temperature inside the reactor system is dependent upon the composition of the carbon containing feed and the desired reaction products. Accordingly, the temperature may be from the melting point of the eutectic mixture of alkali carbonates up to about 1000° C. This may include a temperature from about 250° C. to about 750° C. In some embodiments, the contacting in the process is carried out at a temperature of less than 650° C. This may include, but is not limited to, a temperature of about 600° C. or less, such as about 450° C. to less than 650° C., or at about 600° C.

In the process, the pressures inside the reactor system may be low by comparison to other similar processes. For example, the pressure may be from about 0.5 atm (atmospheres) to about 2 atm, but it is preferably carried out at atmospheric pressure.

In the process, the residence time is a measure of how long the feedstock and products remain within the reaction on an average basis. In some embodiments, the residence time for the process is about 1 minute to about 15 minutes.

The reactor system for the proof of concept was a batch reactor and a stirred tank reactor. However, other suitable reactor configurations are considered such as falling film column reactors, packed column reactor, plate column reactor, spray tower reactor, and a variety of gas-liquid agitated vessel reactors. The process may be carried out as a batch process or in a continuous process.

In one embodiment according to the present invention, the oxidative pyrolysis of polyethylene (PE) and polypropylene (PP) was performed in the presence of a molten salt (a eutectic mixture). The introduction of oxygen to PE-pyrolysis reactions significantly augmented product yields presumably by an increase in radical initiations. This enhancement was most significant under mild reaction conditions (500-600° C.), and the concentration of oxidant (oxygen/feed ratio) was shown to be a key parameter to maximize the formation of desirable hydrocarbon products. Additionally, the application of a molten salt—used as a processing agent—was shown (1) to improve heat transfer to the reaction and (2) to aid in the capture of contaminants (e.g., HCl) while pyrolyzing waste plastic. Ultimately, these advancements look to improve the viability of pyrolysis for the conversion of plastics into chemicals.

The process according to the present invention presents an alternative means of implementing pyrolysis by incorporating a molten salt media and oxygen into the reactor. The addition of oxygen (typically from air; 21% O₂) to the process, possibly through bubbling through the molten salt or other means, further enhances the process through internal heat generation, rendering the overall reaction exothermic (thereby decreasing external heat input requirements), but also increases yields towards light olefins and oils (decreasing waxes and solids) compared to pyrolysis by enhancing (increasing) the radical reactions that can propagate and selectively product light olefins. The process according to the present invention demonstrated here thus shows that the addition of oxygen can be used to increase yields of products at equivalent residence times, thereby reducing reactor size and recycle stream requirements. The process differs from our previously reported “oxycracking” system in that it does not require (but may still include) the addition of a reducible metal oxide, which provides oxygen to the system through a reduction reaction rather than directly through O₂.

The present invention, thus generally described, will be understood more readily by reference to the following examples, which are provided by way of illustration and are not intended to be limiting of the present invention.

While certain embodiments have been illustrated and described, it should be understood that changes and modifications may be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations may be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range may be easily recognized as sufficiently describing and enabling the same range being broken down into at least equal halves, thirds, quarters, fifths, tenths, etc. As a non-limiting example, each range discussed herein may be readily broken down into a lower third, middle third and upper third, etc. As will also be understood by one skilled in the art all language such as “up to,” “at least,” “greater than,” “less than,” and the like, include the number recited and refer to ranges which may be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A process for the cracking of a carbon-containing feedstock to produce olefins, aromatics and/or aliphatic, the process comprising:
contacting, in a reactor system, the carbon-containing feedstock with oxygen gas in the presence of a molten salt matrix consisting of a eutectic mixture of alkali metal carbonates, alkali metal hydroxides, alkali metal nitrates, alkali metal halides, alkaline earth metal carbonates, alkaline earth metal hydroxides, alkali earth nitrates, alkaline earth metal halides, rare earth metal carbonates, transition metal carbonates, transition metal hydroxides, transition metal nitrates, transition metal halides, or a mixture of any two or more thereof, to generate an olefin-containing product stream; and
collecting an olefin from the olefin-containing product stream;
wherein:
the oxygen is fed with the carbon-containing feedstock in a gas stream comprising from greater than 0 wt % to about 21 wt % oxygen in an inert gas; and
the process is conducted in the absence of a catalyst.

2. The process of claim 1, wherein the process is conducted in the absence of a catalyst comprising a transition metal, a transition metal oxide, a rare-earth metal, a rare earth metal oxide, or a combination of any two or more thereof.

3. The process of claim 1, wherein the process is conducted in the absence of a glass-forming oxide.

4. The process of claim 1, wherein the eutectic mixture consists of a mixture of MnCO₃, FeCO₃, CoCO₃, CaCO₃, BaCO₃, Ce₂(CO₃)₃, KOH, NaOH, Ca(NO₃)₂, KNO₃.

5. The process of claim 1 which is a continuous process, a semi-continuous process, or a batch process.

6. The process of claim 1, wherein the reactor system comprises a continuously stirred reactor.

7. The process of claim 1, wherein the olefin comprises a C₂-C₁₂ olefin.

8. The process of claim 1, wherein the olefin comprises ethylene, propylene, 1-butene, 2-methyl-but-1-ene, 1-n-pentene, 1-n-hexene, 2-methyl-pent-1-ene, 3-methyl-pent-1-ene, 1,3-butadiene, 1,3-pentadiene, 1,4-pentadiene, 1,3-hexadiene, 1,4-hexadiene, 1,5-hexadiene, benzene, toluene, ethylbenzene, xylenes, styrene, a—methylstyrene, naphthalene, anthracene, or a combination of any two or more thereof.

9. The process of claim 1, wherein the olefin comprises ethylene or propylene.

10. The process of claim 1, wherein the contacting is conducted at a temperature of less than 650° C.

11. The process of claim 10, wherein the contacting is conducted at a temperature of about 600° C. or less.

12. The process of claim 10, wherein the contacting is conducted at a temperature of about 450° C. to less than 650° C.

13. The process of claim 1, wherein the inert gas comprising helium, argon, or nitrogen.

14. The process of claim 1, wherein the residence time for the process is about 1 minute to about 15 minutes.

15. The process of claim 1, wherein the carbon-containing feedstock is fed to the bottom of the reactor system.

16. The process of claim 1 which is carried out at a pressure of about 0.5 atm to 2 atm.

17. The process of claim 1, wherein the carbon-containing feedstock comprises polyethylene, polypropylene, polyisobutylene, polybutadiene, polystyrene, poly-α-methylstyrene, polacrylates, poly(meth)acrylates, polyvinylchloride, or polyethylene terephthalate.

18. The process of claim 1, wherein the carbon-containing feedstock comprises a refinery range hydrocarbon.

19. The process of claim 18, wherein the refinery range hydrocarbon comprises asphalt, vacuum resid, heavy residual oil, paraffin wax, pyrolysis wax, lubricating oil, diesel, kerosene, naphtha, gasoline, or a combination of any two or more thereof.

20. The process of claim 1, wherein the oxygen is fed to the reactor system in the gas stream comprising from greater than 0 wt % to about 8 wt % oxygen in an inert gas.
